(19) United States
(12) Patent Application Publication
(10) Pub. No.: US 2024/0166588 A1
(43) Pub. Date: May 23, 2024

(54) THE OXIDATION OF CARBON-HYDROGEN BONDS USING OZONE

(71) Applicant: P2 Science, Inc., Woodbridge, CT (US)

(72) Inventors: Patrick FOLEY, New Haven, CT (US); Alexander KENDALL, New Haven, CT (US)

(21) Appl. No.: 18/550,088

(22) PCT Filed: Mar. 10, 2022
(86) PCT No.: PCT/US22/19814

§ 371 Date: Sep. 11, 2023

Related U.S. Application Data
(60) Provisional application No. 63/159,275, filed on Mar. 10, 2021.

(51) Int. Cl. C07C 45/40

(52) U.S. Cl. CPC C07C 45/40; C07C 2601/18; C07C 2602/42

(57) ABSTRACT

The present disclosure is directed to novel methods of oxidizing hydrocarbons, particularly terpenes, using ozone, compounds made according to said methods, and compositions comprising said compounds.

### FIELD OF INVENTION

The present disclosure is directed to novel methods of oxidizing hydrocarbons, particularly terpenes, using ozone, compounds made according to said methods, and compositions comprising said compounds.

### BACKGROUND

The controlled oxidation of hydrocarbons is a difficult chemical process to regulate. The oxidation of C—H bonds in hydrocarbons is particularly challenging because the energetic barriers to such reactions are relatively large, requiring catalysts, expensive reagents, and/or high temperatures. Conditions must be tailored so enough energy is available for an appreciable rate of chemical oxidation while minimizing inevitable but undesirable side reactions.

The oxidation products from terpene hydrocarbons are highly desirable because of their unique chemical architectures and renewable sourcing. Oxidized terpene derivatives constitute one of the most diverse, commercially sought after, and industrially important classes of natural products. Terpenes occur in all organisms and are particularly prevalent in plants, from which they are industrially isolated. The ready commercial access and low-cost of terpenes continually drives innovation into their chemical derivatization which find use in polymer science, the flavor & fragrance industry, the pharmaceutical industry, and as surfactants.

While base terpenes are inexpensive and widely available (C₅ₙH₈ₙ derivatives, n=1, 2, 3, etc.), chemically functionalized terpenes (which require chemical manipulation to acquire) are more useful and exponentially more valuable. Common mono-terpenes available from plant matter, whose oxidation products may be useful, include the follow:

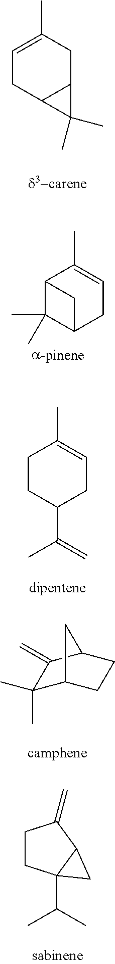

Terpenes may be directly oxidized to yield derivatives such as alcohols, ketones, aldehydes, ethers, and others. These compounds may have variety of diverse uses, including uses as intermediates for the preparation of useful derivatives. While standard organic chemistry techniques are known for oxidizing terpenes (e.g., organic peroxides, oxidation with oxygen (O₂), metal oxidants, hypervalent oxo-halides, etc.), these all suffer from various drawbacks. Oxygen is a commonly used oxidant for these reactions, but the reaction is kinetically hindered because it is a quantum-mechanically spin forbidden reaction (oxygen has a triplet ground state, but alkanes have a singlet ground state).

In addition, typical oxidation conditions generate result in peroxide intermediates, which must then be reduced to yield the desired hydroxy and carbonyl products. Furthermore, existing reaction conditions often require high temperatures, have long reaction times, have poor yields, or require expensive reagents (e.g., stoichiometric metal oxidants). It is also difficult to control these reactions, leading to overoxidation, distributions of structural isomers, and undesired chemical side-reactions and by-products (which can be difficult to remove by traditional purification means).

A few examples have been reported of the direct oxidation of alkanes to alcohols and/or ketones using ozone, but the yields are generally either undetermined (for lack of isolation and purification of a product) or low absent the addition of catalysts. For example, it has been reported that yields of such oxidations may improve in the presence of acidic catalysts, silica gel and/or activated charcoal catalysts, metal catalysts (e.g., copper, manganese and iron), or UV irradiation, but these reactions often lead to cleavage rather than merely C—H bond oxidation.

For example, there is much interest in an economical method of synthesizing linalool from pinene. One currently used method begins with reduction of pinene to pinane, followed by the thermal oxidation of pinane to 2-pinanol and then rearrangement to linalool (As shown below). The critical oxidation step proceeds via free-radical oxidation with oxygen (O₂) to yield 2-pinane hydroperoxide, followed by reduction of the 2-pinane hydroperoxide to yield 2-pinanol. The oxidation typically requires high temperatures (e.g., 80-120° C.) and long durations (e.g., 24 hours) and yet only yields about 12% isolable 2-pinanol (as a mixture of diastereomers, 4:1 cis: trans). See, e.g., Risco, R. & Lemberg, S. Process for producing 2-pinanol. (1973).

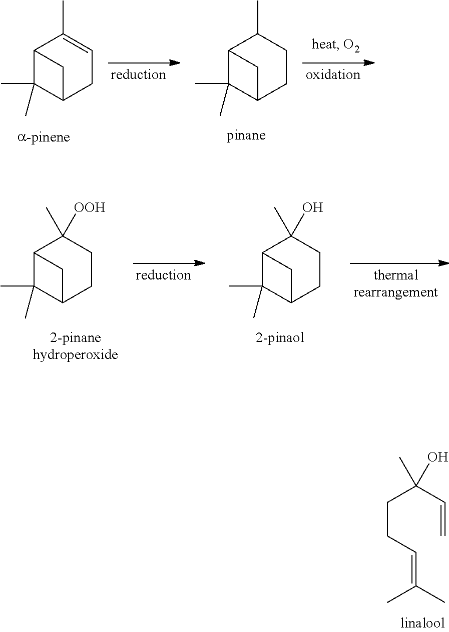

Thus, there is a need for improved commercially viable methods for the direct oxidation of hydrocarbons. Rather, an efficient, fast, selective, and simple (low-cost) process is currently needed for the oxidation of hydrocarbons that doesn't rely on free-radical oxidation with O₂.

### BRIEF SUMMARY OF THE INVENTION

The present disclosure provides a method for the selective oxidation of non-polymeric hydrocarbon moieties using ozone. In a first aspect, the present disclosure provides a method of preparing an alcohol or carbonyl compound from an alkane using ozone, comprising the steps of (1) exposing the alkane to ozone, optionally in an aqueous and/or non-aqueous solvent, and (2) isolating or purifying the resulting alcohol or carbonyl product. Optionally, the method further comprises eliminating the alcohol moiety to yield an alkene, and optionally subsequently reacting the alkene with ozone followed by oxidative or reductive work-up to yield other carbonyl products.

In a second aspect, the present disclosure provides a method of preparing a carbonyl compound from an alkane, comprising the steps of (1) exposing the alkane to ozone, optionally in an aqueous and/or non-aqueous solvent, to form an alcohol, (2) reacting the alcohol with an acid to yield an alkene, (3) exposing the resulting alkene to ozone to yield a secondary ozonide, (d) oxidatively or reductively decomposing the secondary ozonide to yield one or more carbonyl compounds, and (e) isolating or purifying one or more of said carbonyl compounds.

In a third aspect, the present disclosure compounds produced according to the methods disclosed herein. In some embodiments, said compounds are useful as flavor and/or fragrance ingredients in a variety of applications.

### DETAILED DESCRIPTION OF THE INVENTION

The present disclosure provides a method for the selective oxidation of hydrocarbon moieties using ozone. As used herein, “hydrocarbon” refers to a compound comprising a hydrocarbon unit susceptible to oxidation, and this term does not limit the entire structure of said “hydrocarbon” to only hydrogen and carbon atoms. Thus, in one embodiment, the present disclosure provides a method of oxidizing hydrocarbon C—H bonds using ozone, for example, wherein ozone is the sole oxidizing agent.

As used herein the terms “hydrocarbon” and “alkane” are intended to refer to small-molecules, as that term is understood in the art. In contrast, “hydrocarbon” and “alkane” do not refer to macromolecules such as polymers. While polymeric hydrocarbons, such as polyethylene, polypropylene, polystyrene, are technically hydrocarbons, and while polyethylene and polypropylene are both technical entirely aliphatic (i.e., they are alkanes), the present disclosure does not include them within the meaning of the terms “hydrocarbon” and “alkane.” Nevertheless, short oligomers are within the scope of the terms “hydrocarbon” and “alkane” as used herein. While there is no universally recognized cut off for what constitutes a polymer compared to an oligomer, it will be understood that a polymer comprises at least 100 monomeric units. Thus, oligomeric polypropylenes, polyethylenes, polystyrenes, polyethylene glycols, polypropylene glycols, polyesters, polyamides, poly imides, and mixed polymeric and copolymeric compounds having less than 100 monomeric units (in total, for a copolymer) are within the scope of the present invention's hydrocarbons and alkanes.

In some embodiments, the present disclosure provides a method for the selective oxidation of alkane moieties using ozone. As used herein, the term “alkane” does not limit the entire structure of said “alkane” to require complete saturation. Instead, as used herein throughout, “alkane” refers to a compound comprising at least one alkyl group susceptible to oxidation, wherein any other structural element may comprise double bonds, triple bonds and/or aromatic rings. In some embodiments, however, the “alkane” is entirely saturated (i.e., no double bonds, triple bonds or aromatic rings are present). In preferred embodiments, the alkanes are terpenes.

In a first aspect, the present disclosure provides a method (Method 1) of preparing an alcohol or carbonyl compound from an alkane using ozone, comprising the steps of (1) exposing the alkane to ozone, optionally in an aqueous and/or non-aqueous solvent, and (2) isolating or purifying the resulting alcohol or carbonyl product. It is understood that depending on the structure of the starting material alkane, either an alcohol or carbonyl compound (e.g., an aldehyde or ketone) may result from the reaction. In some embodiments, a carboxylic acid may result from the reaction as well, and this is included within the phrase “carbonyl compound” as used herein.

In further embodiments of the first aspect, the present disclosure provides as follows:

1.1 Method 1, wherein the alkane is an alkane according to Formula I:

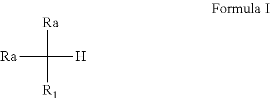

wherein R₁ is either Ra or C(Ra)(Ra)(Ra), and wherein each instance of Ra is independently selected from H, C1-20alkyl, aryl and heteroaryl, and wherein said C1-20alkyl, aryl and/or heteroaryl are each optionally independently substituted, as defined herein, for example, substituted with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

1.2 Method 1.1, wherein the alcohol or carbonyl compound (product) comprises a compound having the formula:

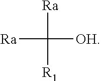

1.3 Method 1.1, wherein R₁ of the alkane is H, i.e., wherein the alkane has the formula:

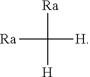

1.4 Method 1.3, wherein the alcohol or carbonyl compound (product) comprises a compound having the formula:

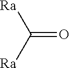

1.5 Method 1.3, wherein the alcohol or carbonyl compound (product) comprises a compound having the formula:

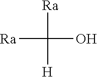

1.6 Method 1.1 wherein one of the Ra moieties, and R₁ of the alkane is H, i.e.,
wherein the alkane has the formula:

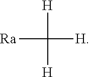

1.7 Method 1.6, wherein the alcohol or carbonyl compound (product) comprises a compound having the formula:

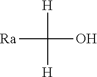

1.8 Method 1.6, wherein the alcohol or carbonyl compound (product) comprises a compound having the formula:

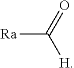

1.9 Method 1.6, wherein the alcohol or carbonyl compound (product) comprises a compound having the formula:

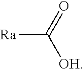

1.10 Any preceding method wherein the alcohol or carbonyl compound (product) comprises any single compound in greater than 50% purity, e.g., in greater than 60% purity, or in greater than 70% purity, or in greater than 80% purity, or in greater than 90% purity, up to 100% purity.

1.11 Any preceding method wherein each instance of Ra is independently selected from H, C1-20alkyl, aryl and heteroaryl, and wherein said C1-20alkyl, aryl and/or heteroaryl are each optionally independently substituted with one or more C1-20alkyl.

1.12 Any preceding method wherein each instance of Ra is independently selected from H and C1-20alkyl, wherein said C1-20alkyl is optionally independently substituted with one or more C1-20alkyl.

1.13 Any preceding method, wherein the alkane is a terpene or terpenoid.

1.14 Any preceding method, wherein the alkane is a hemiterpene, monoterpene, sesquiterpene, diterpene, sesterterpene, triterpene, sesquerterpene, or tetraterpene, or any terpenoid thereof.

1.15 Any preceding method, wherein the alkane is selected from pinene, carene, camphene, bornene, sabinene, elemene, limonene, caryophyllene, valencene, humelene, farnesene, cadinene, and zingiberene.

1.16 Any preceding method wherein the alkane is fully saturated (i.e., the alkane comprises no alkenyl, alkynyl, aryl or heteroaryl moieties).

1.17 Any preceding method, wherein the alkane is a fully saturated hydrocarbon (i.e., the alkane comprises no alkenyl, alkynyl, aryl or heteroaryl moieties, and comprises only carbon and hydrogen atoms).

1.18 Any preceding method, wherein the step (1) of exposing the alkane to ozone comprises exposing the alkane to an ozone/oxygen mixture in the absence of any other oxidants or oxidizing agents.

1.19 Any preceding method, wherein step (1) does not comprise the presence or addition of any catalyst (e.g., any metal, activated charcoal, or silica gel).

1.20 Any preceding method, wherein step (1) occurs in the dark (e.g., the reaction occurs without exposure to light, e.g., UV light).

1.21 Any preceding method, wherein in step (1) the alkane is dissolved or suspended in an aqueous solution or emulsion, optionally in an acidic (i.e., pH <7) or alkaline (e.g., pH >7) aqueous solution or emulsion.

1.22 Method 1.21, wherein the alkane is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).

1.23 Method 1.21, wherein the alkane is dissolved or suspended in an aqueous solution of a sodium, potassium, lithium, calcium or magnesium hydroxide, alkoxide, oxide, carbonate or bicarbonate (e.g., sodium hydroxide or potassium hydroxide).

1.24 Method 1.22 or 1.23, wherein the aqueous solution or emulsion has a pH from 7.5 to 12, or from 8 to 12, or from 9 to 11, or from 9 to 10.

1.25 Any preceding method wherein the alkane is dissolved or suspended in a mixture of an aqueous solution and an organic co-solvent (such as an alcohol, ester, or ether solvent, e.g., methanol, ethanol, propanol, THF, or MTBE).

1.26 Any preceding method, wherein the alcohol or carbonyl compound is obtained directly from the reaction between the alkane and the ozone (e.g., no intermediate partially oxidized or oxidized species are formed or isolated).

1.27 Any preceding method, wherein the method does not comprise the formation of any alkyl peroxide intermediate.

1.28 Any preceding method, wherein the method does not comprise any step comprising a reducing agent between step (1) and step (2).

1.29 Method 1 or any of 1.1-1.27, wherein the method is a batch method.

1.30 Method 1 or any of 1.1-1.27, wherein the method is a continuous flow method, e.g., wherein the method is performed in a flow reactor.

1.31 Method 1 or any of 1.1-1.30, wherein the method is performed in one or more of a falling film reactor, a batch reactor, a continuous stirred-tank reactor, and/or loop reactor, either individually or in series.

1.32 Any preceding method, wherein step (2) comprises separating the alcohol or carbonyl compound product from the reaction solvent, or from the ozone, or both.

1.33 Any preceding method, wherein step (2) comprises distillation, fractional distillation, chromatography, crystallization or a combination thereof.

1.34 Any preceding method, wherein R₁ of the alkane is C(Ra)(Ra)(H), i.e.,
wherein the alkane is an alkane according to Formula II:

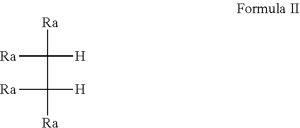

wherein Ra is as defined in any preceding method.

1.35 Method 1.34, wherein the alcohol or carbonyl compound (product) comprises a compound having the formula:

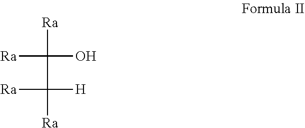

1.36 Method 1.35, wherein the method further comprises the step (3) of exposing the alcohol of the formula to an acid to yield an alkene (e.g., via acid-catalyzed E2 elimination), and optionally isolating or purifying said alkene:

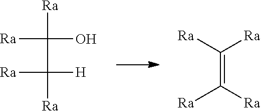

1.37 Method 1.36, wherein said alkene comprises the cis isomer of the alkene or is the trans isomer of the alkene, or a combination thereof.

1.38 Method 1.36, wherein the method further comprises the step (4) of exposing the alkene from step (3) to ozone followed by oxidative and/or reductive decomposition to yield one or two carbonyl compounds (depending on whether the four groups Ra are the same or different):

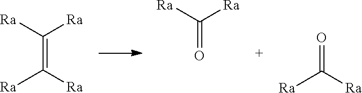

1.39 Method 1.38, further comprising the step (5) of isolating and/or purifying one or both of said carbonyl compounds.

In certain embodiments of Method 1, the compound of Formula I, as used in Method 1, or any of 1.1-1.38, may be selected from any of the following:

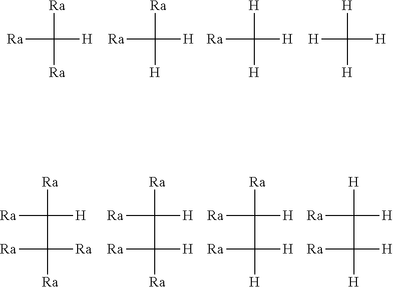

wherein each Ra is independently selected from H, C1-20alkyl, aryl and heteroaryl, and wherein said C1-20alkyl, aryl and/or heteroaryl are each optionally independently substituted, for example, with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

In some embodiments, any one or more Ra groups may be connected to form a ring. For example, the compound of Formula I, as used in Method 1, or any of 1.1-1.38, may be a compound comprising any of the following intramolecular connections:

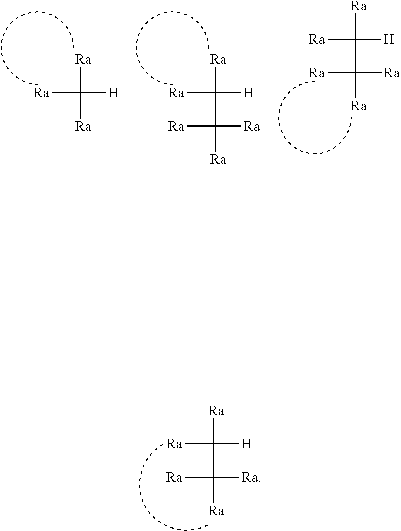

Where an alkene is produced according to Method 1, et seq., the alkene may be reacted with ozone to form a secondary ozonide. The secondary ozonide may be decomposed in-situ, reacted in-situ, or reacted in a subsequent step to yield either oxidized (carboxylic acid and/or ketone) or reduced (aldehyde and/or ketone) products. When more than one compound is the product of any reaction step of Method 1, et seq., any one or more of such products may be the desired product(s), or all may be desired products.

In some embodiments, Method 1, et seq., produces a single desired alcohol compound in admixture with one or more by-product carbonyl compounds, such as a low-molecular weight ketones or aldehydes that may be easily removed by distillation (e.g., formaldehyde, acetaldehyde, acetone, cyclopentanone, or cyclohexanone).

In a second aspect, the present disclosure provides a method (Method 2) of preparing a carbonyl compound from an alkane, comprising the steps of (1) exposing the alkane to ozone, optionally in an aqueous and/or non-aqueous solvent, to form an alcohol, (2) reacting the alcohol with an acid to yield an alkene, (3) exposing the resulting alkene to ozone to yield a secondary ozonide, (d) oxidatively or reductively decomposing the secondary ozonide to yield one or more carbonyl compounds, and (e) isolating or purifying one or more of said carbonyl compounds. For example, Method 2may comprise the steps as follows:

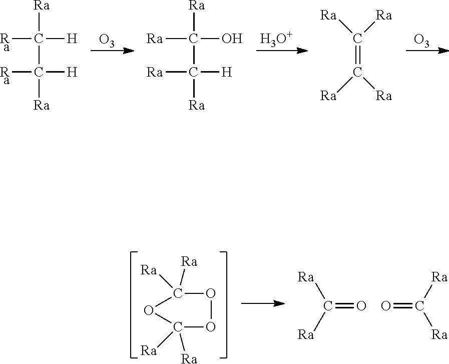

In further embodiments of the second aspect, the present disclosure provides as follows:

2.1 Method 2, wherein the alkane is an alkane according to Formula II:

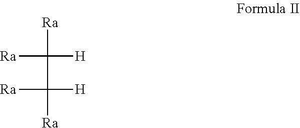

wherein each instance of Ra is independently selected from H, C1-20alkyl, aryl and heteroaryl, and wherein said C1-20alkyl, aryl and/or heteroaryl are each optionally independently substituted, as defined herein, for example, substituted with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

2.2 Method 2.1, wherein the alcohol compound (product) of step (1) comprises a compound having the formula:

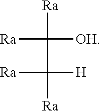

2.3 Method 2.1 or 2.2, wherein the alkene (product) of step (2) comprises a compound having the formula:

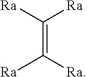

2.4 Method 2.1, 2.2 or 2.3, wherein the carbonyl compound (product) of step (4) comprises one or more compounds having the formula:

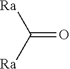

2.5 Any preceding method wherein the carbonyl compound (product) of step (5) comprises any single compound in greater than 50% purity, e.g., in greater than 60% purity, or in greater than 70% purity, or in greater than 80% purity, or in greater than 90% purity, up to 100% purity.

2.6 Any preceding method wherein each instance of Ra is independently selected from H, C1-20alkyl, aryl and heteroaryl, and wherein said C1-20alkyl, aryl and/or heteroaryl are each optionally independently substituted with one or more C1-20alkyl.

2.7 Any preceding method wherein each instance of Ra is independently selected from H and C1-20alkyl, wherein said each of any such C1-20alkyl is optionally independently substituted with one or more C1-20alkyl.

2.8 Any preceding method, wherein the alkane is a terpene or terpenoid.

2.9 Any preceding method, wherein the alkane is a hemiterpene, monoterpene, sesquiterpene, diterpene, sesterterpene, triterpene, sesquerterpene, or tetraterpene, or any terpenoid thereof.

2.10 Any preceding method, wherein the alkane is selected from pinene, carene, camphene, bornene, sabinene, elemene, limonene, caryophyllene, valencene, humelene, farnesene, cadinene, and zingiberene.

2.11 Any preceding method wherein the alkane is fully saturated (i.e., the alkane comprises no alkenyl, alkynyl, aryl or heteroaryl moieties).

2.12 Any preceding method, wherein the alkane is a fully saturated hydrocarbon (i.e., the alkane comprises no alkenyl, alkynyl, aryl or heteroaryl moieties, and comprises only carbon and hydrogen atoms).

2.13 Any preceding method, wherein the step (1) of exposing the alkane to ozone comprises exposing the alkane to an ozone/oxygen mixture in the absence of any other oxidants or oxidizing agents.

2.14 Any preceding method, wherein the alcohol compound (product) of step (1) is obtained directly from the reaction between the alkane and the ozone (e.g., no intermediate partially oxidized or oxidized species are formed or isolated).

2.15 Any preceding method, wherein step (1) does not comprise the formation of any alkyl peroxide intermediate.

2.16 Any preceding method, wherein step (1) does not comprise the presence or addition of any catalyst (e.g., any metal, activated charcoal, or silica gel).

2.17 Any preceding method, wherein step (1) occurs in the dark (e.g., the reaction occurs without exposure to light, e.g., UV light).

2.18 Any preceding method, wherein in step (1) the alkane is dissolved or suspended in an aqueous solution or emulsion, optionally in an acidic (i.e., pH <7) or alkaline (e.g., pH >7) aqueous solution or emulsion.

2.19 Method 2.18, wherein the alkane is dissolved or suspended in an alkaline aqueous solution, optionally wherein the alkaline agent is an inorganic base (e.g., an alkoxide, hydroxide, oxide, carbonate or bicarbonate of an alkali or alkaline earth metal).

2.20 Method 2.18, wherein the alkane is dissolved or suspended in an aqueous solution of a sodium, potassium, lithium, calcium or magnesium hydroxide, alkoxide, oxide, carbonate or bicarbonate (e.g., sodium hydroxide or potassium hydroxide).

2.21 Method 2.19 or 2.20, wherein the aqueous solution or emulsion has a pH from 7.5 to 12, or from 8 to 12, or from 9 to 11, or from 9 to 10.

2.22 Any preceding method wherein the alkane is dissolved or suspended in a mixture of an aqueous solution and an organic co-solvent (such as an alcohol, ester, or ether solvent, e.g., methanol, ethanol, propanol, THF, or MTBE).

2.23 Any preceding method, wherein the method does not comprise any step comprising a reducing agent between step (1) and step (2).

2.24 Any preceding method, wherein steps (3) and (4) of the method occur concomitantly, e.g., in the same reaction vessel simultaneously.

2.25 Method 2.24, wherein the reagent for step (4) is a silicon(II) or sulfur (V) reagent which is stable in the presence of the ozone reagent of step (3).

2.26 Method 2 or any of 2.1-2.25, wherein the method is a batch method.

2.27 Method 2 or any of 2.1-2.25, wherein the method is a continuous flow method, e.g., wherein the method is performed in a flow reactor.

2.28 Method 2 or any of 2.1-2.27, wherein the method is performed in one or more of a falling film reactor, a batch reactor, a continuous stirred-tank reactor, and/or loop reactor, either individually or in series.

2.29 Any preceding method, wherein step (5) comprises separating the carbonyl compound product or products from the reaction solvent, or from the ozone, or both.

2.30 Any preceding method, wherein step (5) comprises distillation, fractional distillation, chromatography, crystallization or a combination thereof.

In certain embodiments of Method 2, the compound of Formula II, as used in Method 2, or any of 2.1-2.30, may be selected from any of the following:

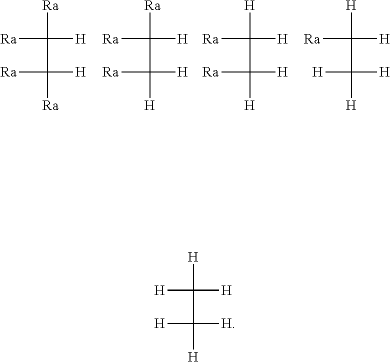

wherein each Ra is independently selected from H, C1-20alkyl, aryl and heteroaryl, and wherein said C1-20alkyl, aryl and/or heteroaryl are each optionally independently substituted, for example, with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

In some embodiments, any one or more Ra groups may be connected to form a ring. For example, the compound of Formula II, as used in Method 2, or any of 2.1-2.30, may be a compound comprising any of the following intramolecular connections:

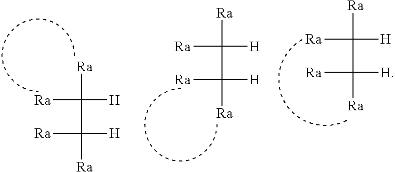

In a third aspect, the present disclosure provides alcohol and carbonyl compounds made according to Method 1, et seq., and or Method 2, et seq. These compounds are useful as flavor or fragrance ingredients. In some embodiments, said compounds are selected from the following:

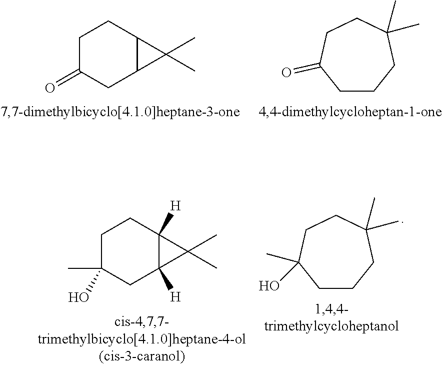

In further aspects, the present disclosure provides compositions comprising the compounds of the third aspect, for example, flavor compositions and/or fragrance compositions.

The term “alkyl” as used herein refers to a monovalent or bivalent, branched or unbranched saturated hydrocarbon group having from 1 to 20 carbon atoms, typically although, not necessarily, containing 1 to about 12 carbon atoms, such as methyl, ethyl, n-propyl, isopropyl, n-butyl, isobutyl, t-butyl, octyl, and the like. The term alkyl also may include cycloalkyl groups. Thus, for example, the term C6 alkyl would embrace cyclohexyl groups, the term C5 would embrace cyclopentyl groups, the term C4 would embrace cyclobutyl groups, and the term C3 would embrace cyclopropyl groups. In addition, as the alkyl group may be branched or unbranched, any alkyl group of n carbon atoms would embrace a cycloalkyl group of less than n carbons substituted by additional alkyl substituents. Thus, for example, the term C6 alkyl would also embrace methylcyclopentyl groups, or dimethylcyclobutyl or ethylcyclobutyl groups, or trimethylcyclopropyl, ethylmethylcyclopropyl or propylcyclopropyl groups.

The term “alkenyl” as used herein refers to a monovalent or bivalent, branched or unbranched, unsaturated hydrocarbon group typically although not necessarily containing 2 to about 12 carbon atoms and 1 -10 carbon-carbon double bonds, such as ethylene, n-propylene, isopropylene, n-butylene, isobutylene, t-butylene, octylene, and the like. In like manner as for the term “alkyl”, the term “alkenyl” also embraces cycloalkenyl groups, both branched an unbranched with the double bond optionally intracyclic or exocyclic.

The term “alkynyl” as used herein refers to a monovalent or bivalent, branched or unbranched, unsaturated hydrocarbon group typically although not necessarily containing 2 to about 12 carbon atoms and 1-8 carbon-carbon triple bonds, such as ethyne, propyne, butyne, pentyne, hexyne, heptyne, octyne, and the like. In like manner as for the term “alkyl”, the term “alkynyl” also embraces cycloalkynyl groups, both branched an unbranched, with the triple bond optionally intracyclic or exocyclic.

The term “aryl” as used herein refers to an aromatic hydrocarbon moiety comprising at least one aromatic ring of 5-6 carbon atoms, including, for example, an aromatic hydrocarbon having two fused rings and 10 carbon atoms (i.e., a naphthalene).

By “substituted” as in “substituted alkyl,” “substituted alkenyl,” “substituted alkynyl,” and the like, it is meant that in the alkyl, alkenyl, alkynyl, or other moiety, at least one hydrogen atom bound to a carbon atom is replaced with one or more non-hydrogen substituents, e.g., by a functional group.

The terms “branched” and “linear” (or “unbranched”) when used in reference to, for example, an alkyl moiety of C a to Cb carbon atoms, applies to those carbon atoms defining the alkyl moiety. For example, for a C4 alkyl moiety, a branched embodiment thereof would include an isobutyl, whereas an unbranched embodiment thereof would be an n-butyl. However, an isobutyl would also qualify as a linear C3 alkyl moiety (a propyl) itself substituted by a Ci alkyl (a methyl).

Examples of functional groups include, without limitation: halo, hydroxyl, sulfhydryl, C₁-C₂₀alkoxy, C₂-C₂₀ alkenyloxy, C₂-C₂₀ alkynyloxy, C₅-C₂₀ aryloxy, acyl (including C₂-C₂₀ alkylcarbonyl (—CO-alkyl) and C₆-C₂₀ arylcarbonyl (—CO-aryl)), acyloxy (—O-acyl), C₂-C₂₀ alkoxycarbonyl (—(CO)—O-alkyl), C₆-C₂₀ aryloxycarbonyl (—(CO)—O-aryl), halocarbonyl (—CO)—X where X is halo), C₂-C₂₀ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-substituted C₁-C₂₀ alkylcarbamoyl (—(CO)—NH(C₁-C₂₀ alkyl)), di-substituted alkylcarbamoyl (—(CO)—N(C₁-C₂₀ alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₀ alkyl)-substituted amino, mono- and di-(C₅-C₂₀ aryl)-substituted amino, C₂-C₂₀ alkylamido (—NH—(CO)-alkyl), C₅-C₂₀ arylamido (—NH—(CO)-aryl), imino (—CR═NH where R=hydrogen, C₁-C₂₀ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₀ alkylsulfanyl (—S-alkyl; also termed “alkylthio”), arylsulfanyl (—S-aryl; also termed “arylthio”), C₁-C₂₀ alkylsulfinyl (—(SO)-alkyl), C₅-C₂₀ arylsulfinyl (—(SO)-aryl), C₁-C₂₀ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂), phosphino (—PH₂), mono- and di-(C₁-C₂₀ alkyl)-substituted phosphino, mono- and di-(C₅-C₂₀ aryl)-substituted phosphino; and the hydrocarbyl moieties such as C₁-C20 alkyl (including C₁-C₁₈ alkyl, further including C₁-C₁₂ alkyl, and further including C₁-C₆ alkyl), C₂-C₂₀ alkenyl (including C₂-C₁₈ alkenyl, further including C₂-C₁₂ alkenyl, and further including C₂-C₆ alkenyl), C₂-C₂₀ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₂₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. For example, the alkyl or alkenyl group may be branched. For example, the “substituent” is an alkyl group, e.g., a methyl group.

Suitable solvents and reactions conditions (concentration, time, temperature) for the ozonolysis step (3) of Method 2, et seq., are known to those in the art and are not limited in any way in the present disclosure.

### Typical Conditions for Method 1

Suitable solvents for step (1) of any of Method 1 et seq. include apolar, polar protic and/or polar aprotic solvents, for example alcoholic solvents (e.g., methanol, ethanol, propanol, isopropanol, butanol). In some embodiments, the solvent for step (1) of Method 1, et seq., comprises an aqueous solution or emulsion, optionally an aqueous alkaline or aqueous acidic solution or emulsion. Any such aqueous solution may be a buffer. A buffer may be employed to maintain a pH >7. In some embodiments, the pH is between 7.5 and 12, or between 8 and 12, or between 9 and 11 or between 9 and 10. In some embodiments, the reaction occurs in an aqueous layer that forms an emulsion upon mixing with an organic layer. The organic layer may be the alkane substrate and/or a solution of the alkane substrate in an organic solvent. In a preferred embodiment, an alkaline aqueous solution is combined with the near alkane, such as, for example, a2 M NaOH solution mixed 1:1 with neat alkane.

In some embodiments, the alkane contains heteroatoms. In a preferred embodiment, terpene derivatives, organophosphorus, and organosulfur species are the alkane.

In some embodiments, the reaction is carried out at a temperature of −25° C. to 200° C. In a preferred embodiment, the reaction is run at 5° C. In some embodiments, the reaction is carried out for 0.1 to 100 hours. In a preferred embodiment the reaction is run for 2 hours.

In some embodiments, the ozonation is combined with electromagnetic irradiation to promote reactivity. In some embodiments, the wavelength is between 100-1000 nm, with a preferred embodiment between 200-280 nm. In other embodiments, the ozonation reaction is not exposed to any UV light, or is not exposed to any light (i.e., the reaction is in the dark).

### Typical Conditions for Method 2

Suitable solvents for step (1) of Method 2 et seq. are as described for step (1) of Method 1 et seq. Suitable solvents for the steps (2) to (4) of any of Method 2 et seq. include apolar, polar protic and/or polar aprotic solvents, for example alcoholic solvents (e.g., methanol, ethanol, propanol, isopropanol, butanol), or acidic solvents (e.g., formic acid, acetic acid, propionic acid) are used. In some embodiments, a buffer may be employed to maintain a pH <7. In some embodiments the buffer is in an aqueous layer that forms an emulsion upon mixing. In a preferred embodiment, propionic acid used 1:1 with neat alkane for step (1). In some embodiments a reagent is added before the ozonation step (3) to react with the secondary ozonide in-situ as it is formed. This results in formation of the final carbonyl species in a combination of steps (3) and (4) in single reaction vessel. In other embodiments, the secondary ozonide is decomposed exogenously, i.e., as a separate step (4) conducted in a different vessel or at a different time than step (3). In a preferred embodiment, silicon(II) or sulfur(IV) reagents are used to react the secondary ozonide in-situ into carbonyl species.

Suitable silicon(II) reagents for practice of the present invention include dialkoxysilyl hydride species, such as compounds of the general formula HSi(R¹)(R²)(R³), wherein R¹, R² and R³ are independently selected from optionally substituted C₁₋₆alkyl, halogen, or alkoxy, optionally substituted C₁₋₆alkenyl, and optionally substituted aryl or heteroaryl. In some embodiments, R² and R³ are the same.

Suitable sulfur(IV) reagents for practice of the present invention include dialkyl sulfoxides of the formula R¹—(S═O)—R², wherein R¹ and R² are independently optionally substituted C₁₋₆alkyl. In some embodiments, R¹ and R² are independently selected from methyl, ethyl, propyl, isopropyl, n-butyl, s-butyl, t-butyl, and isobutyl. In some embodiments, R¹ and R² are the same. In some embodiments, the sulfur(IV) reagent is dimethyl sulfoxide (DMSO).

### Selectivity of Alkane Oxidation

Ozone-mediated direct oxidation (ozonation) of alkanes has been found by the inventors to have high selectivity based on both thermodynamic factors (bond dissociation energy, stabilization of reaction intermediates) and kinetic factors (steric hinderance). For instance, a cyclohexane ring is oxidized at the equatorial C—H position eight times faster than at the axial position. Bridgehead C—H bonds are particularly averse to oxidation, while fused ring C—H bonds can react if the rings don't contain strain. Thus, selectivity is high for: (1) low bond-dissociation energy C—H sites, (2) tertiary C—H>secondary C—H>primary C—H, (3) planarizable carbon (sp³ to sp² conformation allowed), and (4) minimum steric hinderance. These factors lead to the chemoselectivity observed for these oxidations.

Without being bound by theory, these observations suggest a mechanism in which the intermediate species formed is a carbon-based cation or radical. Notably, any radical character at the carbon ultimately would lead to alkene formation via a carbon-centered radical and hydroxide radical that are in a triplet state (thus unable to recombine to form an alcohol). It can be assumed that a radical pathway is a minor process for three reasons: (1) a small amount of alkene ozonolysis products are observed with the same ratios regardless of pH or reaction medium, suggesting an acid independent unimolecular (or caged) pathway to alkene formation (as a minor product); (2) there is almost no peroxide buildup during the reaction (which would be the likely fate of the carbon-based radical and hydroxide radical if they diffused through the solvent cage); and (3) in solution—particularly polar media—ozone conventionally operates predominantly via 2-electron pathways and zwitterionic intermediates. Thus, it is likely the resonance form of the intermediate formed dominates as a singlet-state zwitterion as opposed to a singlet-state diradical species which leads to alcohol formation as the major product. Therefore, without being bound by theory, it is believed that the direct oxidation of alkane C—H bonds via ozone proceeds according to the following mechanism:

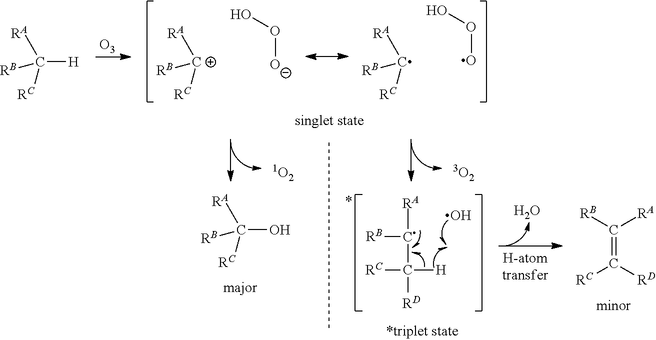

For example, it is found that when oxidizing pinane (as a mixture of cis- and trans-isomers), the only observable alcohol product is cis-2-pinanol. There is a concomitant enrichment of the trans-pinane relative to cis-pinane, showing that the oxidation is selective for cis-pinane oxidation to cis-2-pinanol. In the analogous reaction with O₂ and pinane, there is still selectivity for cis-pinane oxidation, however the selectivity for cis-2-pinanol: trans-2-pinanol is ˜6:1. It is likely that both the high temperatures and radical intermediates for O₂ oxidation contribute to the lower chemoselectivity than is observed in the ozonation of pinane at lower temperatures (>20:1).

### EXAMPLES

Example 1. A mixture containing 10 g pinane (2:1 cis:trans) and 55 mL of 2 M NaOH (aqueous) is sparged with ozone (35% O₃, air, 3 LPM) with vigorous stirring for 90 minutes at 5° C. The reaction mixture is separated by allowing the two phases to separate and then decanting the organic layer from the aqueous layer. The organic layer is tested and found to contain 2 mmol/L peroxides and the aqueous layer does not contain detectible peroxides (as determined by iodometric titration). The organic layer is washed with brine, separated, and decanted, to yield 5.0 g of organic material that consists of cis-2-pinanol, trans-pinane, cis-pinane, nopinone, and traces of minor products (<5%). 6 grams of material consisting of primarily cis- and trans-pinane with various minor oxidation products and 1 mL of water is also recovered from an in-line trap. The basic aqueous layer is also found to contain ring opened oxidation products totaling ˜3% of the mass.

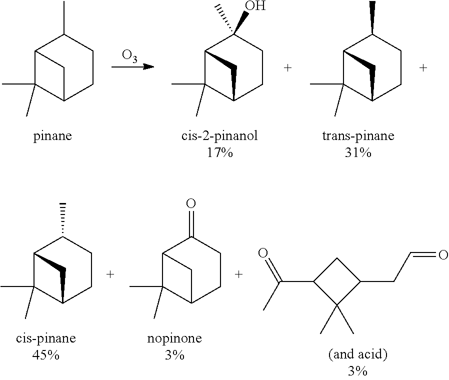

Example 2. A mixture containing 10 g cis-carane and 1,1,4-trimethylcycloheptane (3:2) and 55 mL of 2 M NaOH (aqueous) is sparged with ozone (35% O₃, air, 3 LPM) with vigorous stirring for 90 minutes at 5° C. The reaction mixture is separated by allowing the two phases to separate and decanting the organic layer from the aqueous layer. The organic layer is found to contain <1 mmol/L peroxides and the aqueous layer does not contain detectible peroxides (as determined by iodometric titration). The organic layer is washed with brine, separated, and decanted, to yield 7.5g of organic material that consists of cis-carane, 1,1,4-trimethylcycloheptane, with an estimated 2-4% by mass of the desired oxidation products cis-caran-3-ol and 7,7-dimethylbicyclo[4.1.0]haptan-3-one. The basic aqueous layer contains ring opened oxidation products totaling <0.5% of the mass.

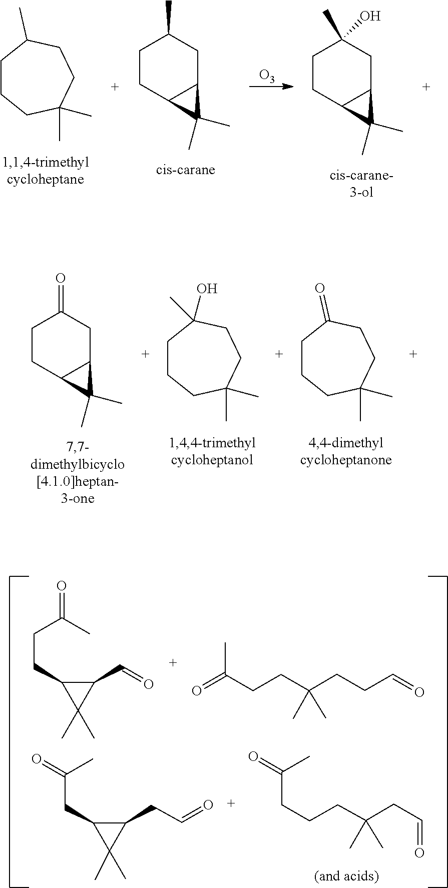

The Examples provided herein are understood to be exemplary only, and in no way limit the scope of the invention described or claimed herein.

1. A method of oxidizing hydrocarbon C—H bonds using ozone.

2. A method of preparing an alcohol or carbonyl compound from an alkane using ozone, comprising the steps of (1) exposing the alkane to ozone, optionally in an aqueous and/or non-aqueous solvent, and (2) isolating or purifying the resulting alcohol or carbonyl product.

3. The method of claim 2, wherein the alkane is an alkane according to Formula I. 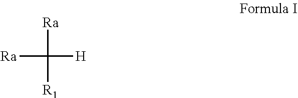
wherein R₁ is either Ra or C(Ra)(Ra)(Ra), and wherein each instance of Ra is independently selected from H, C1-20alkyl, aryl and heteroaryl, and wherein said C1-20alkyl, aryl and/or heteroaryl are each optionally independently substituted, as defined herein, for example, substituted with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

4. The method according to claim 3, wherein R₁ of the alkane is C(Ra)(Ra)(H), and the alkane is an alkane according to Formula II: 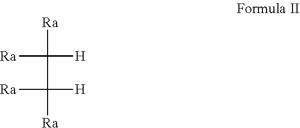
wherein Ra is independently selected from H, C1-20alkyl, aryl and heteroaryl, and
wherein said C1-20alkyl, aryl and/or heteroaryl are each optionally independently substituted, as defined herein, for example, substituted with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

5. The method according to claim 4, wherein the method further comprises the step (3) of exposing the alcohol of the formula to an acid to yield an alkene (e.g., via acid-catalyzed E2 elimination), and optionally isolating or purifying said alkene.

6. The method according to claim 5, wherein the method further comprises the step (4) of exposing the alkene from step (3) to ozone followed by oxidative and/or reductive decomposition to yield one or two carbonyl compounds.

7. A method of preparing a carbonyl compound from an alkane, comprising the steps of (1) exposing the alkane to ozone to form an alcohol, (2) reacting the alcohol with an acid to yield an alkene, (3) exposing the resulting alkene to ozone to yield a secondary ozonide, (d) oxidatively or reductively decomposing the secondary ozonide to yield one or more carbonyl compounds, and (e) isolating or purifying one or more of said carbonyl compounds.

8. The method according to claim 7, wherein the alkane is an alkane according to Formula II: 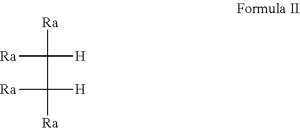
wherein each instance of Ra is independently selected from H, C1-20alkyl, aryl and heteroaryl, and wherein said C1-20alkyl, aryl and/or heteroaryl are each optionally independently substituted, as defined herein, for example, substituted with one or more C1-20alkyl, hydroxy, C1-20alkoxy, carboxy, halogen, cyano, acyl (C1-20alkylcarbonyl), C1-20alkoxycarbonyl, or C1-20alkylaminocarbonyl.

9. The method according to claim 1, wherein step (1) occurs in aqueous and/or non-aqueous solvent.

10. The method according to claim 9, wherein the step (1) occurs in an alkaline aqueous solution or alkaline aqueous emulsion.

11. The method according to claim 1, wherein step (1) does not comprise the presence or addition of any catalyst.

12. The method according to claim 1, wherein in step (1) ozone is the only oxidizing agent present.

13. The method according to claim 1, wherein the alkane is a terpene or terpenoid, optionally, wherein the alkane is a fully saturated terpene or terpenoid.

14. An alcohol or carbonyl compound prepared according to claim 1.

15. The alcohol or carbonyl compound according to claim 14, wherein the alcohol or carbonyl compound is selected from the group consisting of:

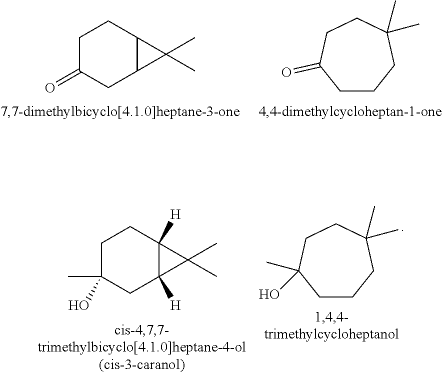

16. A flavor or fragrance composition comprising a compound according to claim 15.
